(19) United States
(12) Patent Application Publication
(10) Pub. No.: US 2007/0164576 A1
(43) Pub. Date: Jul. 19, 2007

(54) Contact lens device for applying and removing a contact lens

(76) Inventor: Stanton Kim, Gilroy, CA (US)

Correspondence Address: DR. STANTON KIM, 10760 GREEN VALLEY DR, GILROY, CA, 95020, US

(21) Appl. No.: 11/334,916

(22) Filed: Jan. 18, 2006

(51) Int. Cl. A61F 9/00

(52) U.S. Cl. 294/1.2

(57) ABSTRACT

A contact lens device comprising a lens support having a concave surface that is treated or modified to hold a contact lens. A handle or stem is coupled to the lens support for positioning the lens support during the process of inserting or removing a contact lens from a wearer's eye.

### FIELD OF THE INVENTION

The present invention relates generally to a soft contact lens device used for insertion and removal of a soft contact lens to or from the surface of an eye.

### BACKGROUND

Contact lenses can be made to correct most visual conditions correctable by regular eyeglasses. Moreover, contact lenses are not readily visible and provide the user with a wider field of vision than do eyeglasses. The first contact lenses were of the “hard” variety, that is to say, they are lathe cut and polished or are molded of a relatively rigid material, and formed by heat and pressure to the shape of a wearer's or user's eye. The user must learn to wear them over a period of time. Many persons have experienced difficulty in wearing hard contact lenses.

A recent innovation has been the “soft” contact lens constructed of flexible, liquid absorbent, and gas permeable material. Although soft contact lenses are comfortable to wear, the soft contact lens has some problems, including difficulty of removal. Contact lens applicators or removers are generally known and early applicators were designed primarily for the application of rigid contact lenses. Such known devices have included holders, suction apparatus, spring-loaded devices, lighting devices and eyepieces.

Generally, soft contact lenses are removed by the wearer moving the contact lens off the cornea onto the sclera and pinching the soft contact between his fingertips, thereby removing the contact lens from the eye. Unfortunately, some people have difficulty in removing the contact lens because of their difficulty in placing their fingers on the cornea, while others have long or sharp fingernails and still others are generally clumsy having large fingers.

One type of contact removing device uses a tweezers action. Examples of tweezers type contact lens removers are in U.S. Pat. No. 4,192,204 to Feldman entitled “Soft Contact Lens Apparatus” and in U.S. Pat. No. 4,986,586 to Eilrich et al. entitled “Device for Removing Soft Contact Lens.” By holding a tweezers device in either hand, using the thumb and the fore finger, the device is brought up to the eye having a contact lens. Using the tweezers, gentle pressure is put onto the contact lens with the tips, and the device is squeezed together in a tweezers motion. The contact lens is gripped and gently pulled away from the eye, removing the contact lens. Tweezers type devices may be used to successfully remove a soft contact lens however, a tweezers device may also be prone to cause damage to a soft contact lens.

Other devices may be described generally as cup or suction devices. A suction or capillary action is developed between the lens and the device for holding the lens in a desired position during the application or removal of a lens. For example, U.S. Pat. No. D310,380 to England entitled “Contact Lens Remover” uses a cup design, however, only the removal of a contact lens is facilitated. In U.S. Pat. No. 4,167,283 to Feldman entitled “Apparatus for Applying a Soft Contact Lens” also uses a cup design however, only facilitates the application of a soft contact lens.

Generally, soft contact lenses are first inserted and then later removed by the wearer. A soft contact lens may be removed from the wearer's eye by moving the contact lens off the cornea onto the sclera and pinching the soft contact between his fingertips. Unfortunately, some people have difficulty in inserting or removing the contact lens. As discussed above, contact lenses may be removed by using a scissoring or tweezers device, or by a using a cup type device. A scissoring action is inappropriate for soft contact lenses, and cup devices may be used to either singularly apply or singularly remove a contact lens. Many contact lens devices are only able to perform a singular function of either inserting or removing a contact lens, but do not perform both functions.

### SUMMARY OF THE INVENTION

A contact lens device comprising a lens support with a concave surface. The concave surface is treated or modified for adhering to and holding a contact lens. A handle or stem is coupled to the lens support for positioning the lens support during the process of inserting or removing a contact lens from a wearer's eye.

### BRIEF DESCRIPTION OF THE DRAWINGS

FIG. 1 is a cross section of an exemplary contact lens device.

FIG. 2A is a cross section of an alternate embodiment of a contact lens device.

FIG. 2B is a cross section of another embodiment of a contact lens device.

FIG. 2C is an axially rotated embodiment of an AA view of the FIG. 1 cross-section.

FIG. 2D is another embodiment of an AA view of the FIG. 1 cross-section.

FIG. 3 is a magnified cross sectional view of an exemplary contact lens device of FIG. 1.

### DETAILED DESCRIPTION

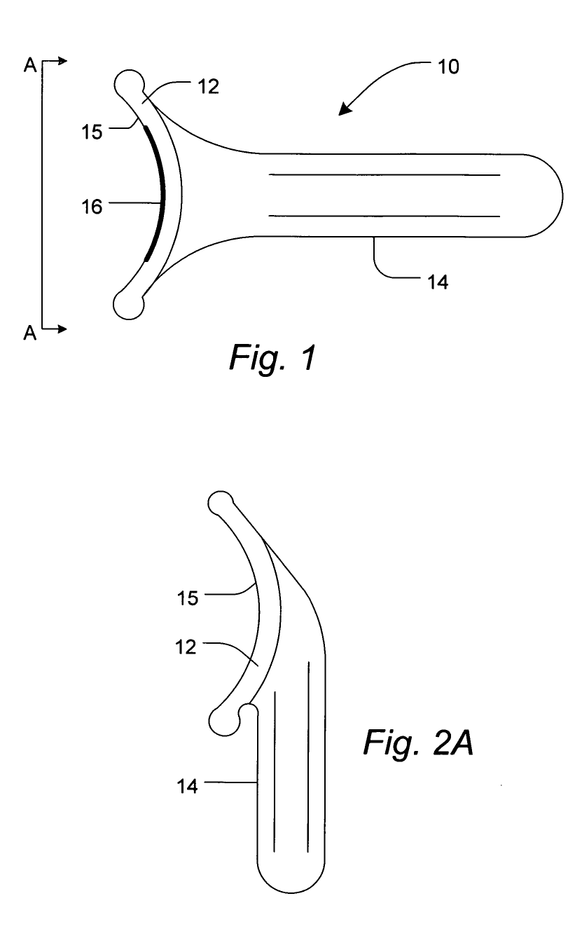

Referring to FIG. 1, an exemplary contact lens device 10 may be used to remove a contact lens from a wearer's eye. A handle or stem 14 is coupled to a lens support 12. The handle or stem 14 is held by either the wearer of the contact lens or an assistant to aid in positioning the lens support 12 during the insertion or removal of a contact lens onto or from the wearer's eye.

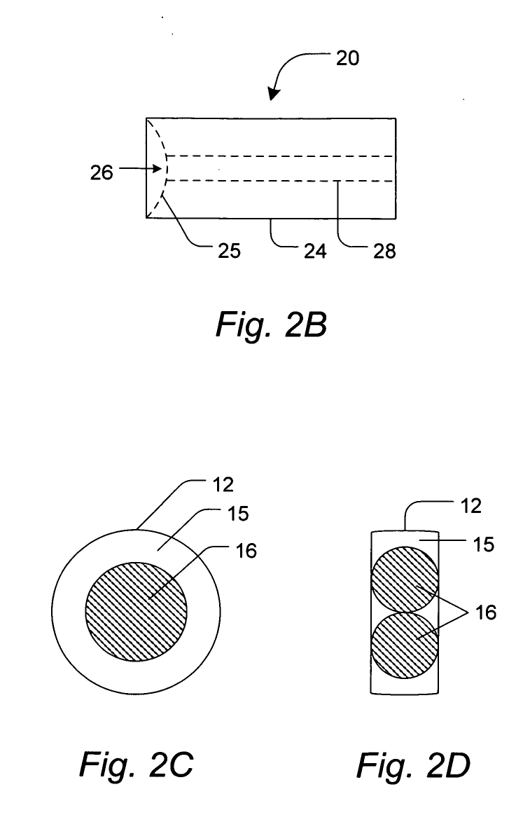

The handle or stem 14 may be axially aligned with the lens support 12 as shown in FIG. 1. Referring to FIG. 2A, the handle or stem 14 may be alternately coupled to the lens support 12 at a variety of angles. Referring to FIG. 2B,. in an alternate embodiment, a contact lens device 20 having a handle or stem 24 may also exist in a variety of shapes, dimensions, and proportions with respect to a lens support 25. A handle or stem and/or lens support are generally comprised of or made of flexible materials such as rubber, Styrofoam, plastics, or sponge materials. The flexible materials used are sufficiently resilient to permit deflection of the lens support, or handle or stem, upon application of the lens to the eye to ensure a secure continuous, surface-to-surface contact between the lens and the surface of the eye without injury to the eye or damage to the lens.

Referring again to FIG. 1, the lens support 12 has a concave surface 15 capable of holding a contact lens. Generally, the concave surface 15 of the lens support 12 has or a radius of curvature between 4 and 11 millimeters (mm) to approximately conform to the curvature of a wearer's eye or an average human eye. Also, the concave surface 15 of the lens support 12 has a lens contact area or a lens contact diameter that is smaller than the outside diameter of a contact lens. The contact dimension is approximately between 4 and 14 mm.

Referring to FIG. 2B, a concave surface 25 of a lens support may have a solid or continuous surface, or optionally may have a non-continuous surface having a single opening or a plurality of openings. A non-continuous surface may be used to vent, prevent, or control the suction or capillary action between the concave surface 25 and a contact lens. A contact lens device 20 having a concave surface 25, for example, has a single opening 26 in the concave surface 25 that extends 28 through the handle or stem 24.

Referring again to FIG. 1, in one embodiment of a contact lens device, the cross section of FIG. 1 is axially rotated to form a round lends support 12 member having a concave surface approximately resembling an arc portion of a sphere. Also referring to FIG. 2C, the front view (from AA in FIG. 1) looks into the round cup of a lens support 12, having a concave surface 15, and a treated or modified surface 16. Alternatively, referring to FIG. 2D, an alternate embodiment of a contact lens device generally has a flat shape. The front view (from AA in FIG. 1) of the alternate embodiment lens support 12, has a concave surface 15, and a treated or modified surface 16.

Referring again to FIG. 1, the concave surface 15 of the lens support 12 is treated or modified to provide a gripping or adhesive property that overcomes a contact lens-to-eye surface tension during removal of a contact lens. In one embodiment, the modified surface 16 is comprised of an applied adhesive material that is pre-applied. Optionally, the adhesive material may be applied by the user of the contact lens device. For example, the contact lens device may be dipped into an adhesive material or an adhesive solution by the wearer of the contact lens prior to removing a contact lens from the eye. In another embodiment, the concave surface 15 may be treated or modified to comprise a surface pattern. For example, a fish-scale pattern or angled protrusions may be configured on the concave surface 15 of the lens support 12.

Additionally, the entire concave surface 15 or a portion of the concave surface 15 may be treated or modified. When a portion of the concave surface 15 is treated or modified, a variety of areas or patterns of adhesive material may be applied to the concave surface 15 of the lens support 14. Referring to FIG. 2C, a single circle pattern is used in one embodiment. In alternative exemplary embodiments, a ring pattern, a plurality of concentric rings, an arrangement of circles as shown in FIG. 2D, or an arrangement of distributed or patterned circles may be incorporated.

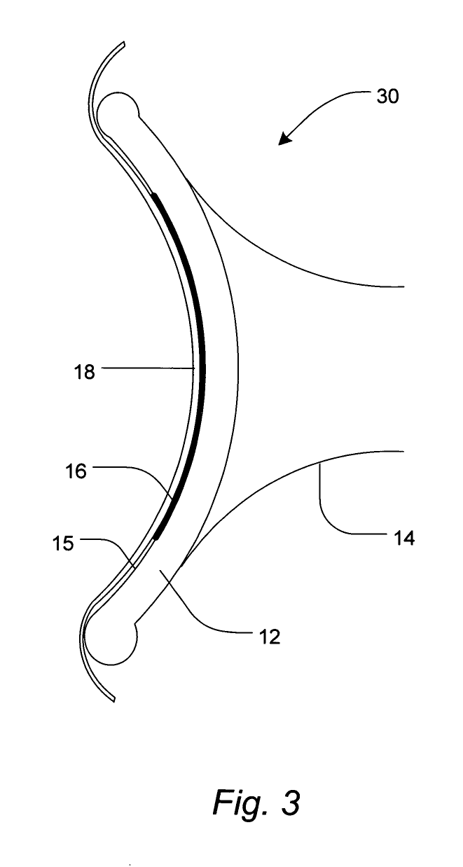

Referring to FIG. 3, a protective layer 18 is coupled to or contacts the treated or modified surface 16 of the lens support 14. The protective layer 18 functionally protects or isolates the treated or modified surface 16. Generally, the protective layer 18 is held in place by the gripping or adhesive property of the treated or modified surface 16 and may have a solid or continuous surface or may optionally have a non-continuous surface with a single opening or a plurality of openings. The protective layer may be formed to cover the entire concave surface, or may cover a portion of the concave surface 15. If a portion of the concave surface 15 is covered, the entire modified surface 16 or a portion of the modified surface 16 may be optionally covered by the protective layer 18.

The surface of the protective layer 18 has a capillary action property that is sufficient to hold or grasp a contact lens, and also to release and transfer a contact lens when the contact lens comes in contact with a wearer's eye during a contact lens insertion procedure. During the insertion procedure, the contact lens-to-eye surface tension overcomes the capillary action or gripping action of the protective layer 18.

Optionally, the protective layer 18 may be formed to have a varying or non-uniform thickness. The (non-uniform or varying) thickness may provide a curvature or profile that is different than the curvature and shape of the concave surface 15. The thickness of the protective layer 18 may be formed to control the gripping properties of the protective layer 18 in comparison to curvature and shape of an unprotected concave surface 15 having a treated or modified surface 16.

Generally, the contact lens device 30 having a protective layer 18 is used to hold a contact lens prior to inserting the lens into a wearer's eye. The contact lens is carefully brought toward and in contact with the wearer's eye. The contact lens device 30 releases and transfers the contact lens to the wearer's eye when the contact lens comes in contact with the eye.

After a contact lens has been inserted into a wearer's eye, the contact lens device 30 may then be used to remove a contact lens from the wearer's eye. The protective layer 18 is removed from the contact lens device 30, thereby exposing the modified surface 16 of the lens support 12. The concave surface 15 of the lens support 12 is then carefully placed so that the treated or modified surface 16 of the lens support 12 comes into contact with the contact lens in the wearer's eye. The contact lens device 30 grips or adheres to the contact lens, overcoming the contact lens-to-eye surface tension, and then removes the contact lens as the contact lens device 30 is withdrawn from the wearer's eye.

The present invention is not to be limited in scope by the specific embodiments described herein. Indeed, various modifications of the invention in addition to those described herein will become apparent to those skilled in the art from the foregoing description and accompanying figures, and such modifications are intended to fall within the scope of the appended claims.

1. A contact lens device comprising:
a lens support having a concave surface for holding a contact lens;
an adhesive material coupled to the concave surface of the lens support, the adhesive material being configured to adhere to a contact lens to overcome a lens-to-eye capillary attraction during a contact lens removal procedure; and
a stem member coupled to the lens support.

2. The contact lens device of claim 1, wherein the lens support surface treatment comprises the application of an adhesive material to the concave surface of the lens support.

3. The contact lens device of claim 2, wherein the application of an adhesive material to the concave surface of the lens support of the contact lens device is performed by the user of the contact lens device.

4. The contact lens device of claim 1, further comprising a removable protective cover applied to the treated surface of the lens support.

5. The contact lens device of claim 4, wherein the removable protective cover is applied to the contact lens device after an adhesive material has been applied to the concave surface of the lens support.

6. The contact lens device of claim 4, wherein the removable protective cover has a non-uniform thickness.

7. The contact lens device of claim 1, wherein the lens support comprises a flexible material.

8. The contact lens device of claim 1, wherein the stem member comprises a flexible material.

9. The contact lens device of claim 1, wherein the concave surface of the lens support has a lens contact area which is smaller than the area of a contact lens.

10. The contact lens device of claim 1, wherein the concave surface of the lens support has a lens contact diameter between approximately 4 and 14 millimeters.

11. The contact lens device of claim 1, wherein the concave surface of the lens support has an approximate radius of curvature between 4 to 11 millimeters.

12. The contact lens device of claim 1, further comprising an opening formed in the lens support to reduce a suction or capillary effect therein which holds a contact lens in the lens support during the application of a contact lens.

13. The contact lens device of claim 1, wherein the stem member comprises a tubular construction having a longitudinal opening aligned with an opening in the lens support.

14. A contact lens device comprising:
a lens support having a concave surface;
an adhesive material coupled to the concave surface of the lens support, the adhesive material being configured to adhere to a contact lens and overcome a contact lens-to-eye capillary attraction during a contact lens removal procedure;
a removable protective cover applied over the adhesive material of the lens support, the removable protective cover and lens support being configured to hold a contact lens; and
a stem member coupled to the lens support.

15. The contact lens device of claim 14, wherein the application of an the adhesive material to the concave surface of the lens support is performed by the user of the contact lens device.

16. The contact lens device of claim 14, wherein the removable protective cover has a non-uniform thickness.

17. The contact lens device of claim 14, wherein the lens support comprises a flexible material.

18. The contact lens device of claim 14, wherein the stem member comprises a flexible material.

19. The contact lens device of claim 14, wherein the concave surface of the lens support has a lens contact area which is smaller than the area of a contact lens.

20. The contact lens device of claim 14, wherein the concave surface of the lens support has a lens contact diameter between approximately 4 and 14 millimeters.

21. The contact lens device of claim 14, wherein the concave surface of the lens support has an approximate radius of curvature between 4 to 11 millimeters.

22. The contact lens device of claim 14, further comprising an opening formed in the lens support, the opening being configured to reduce a suction or capillary effect therein which would tend to hold a contact lens in the lens support during the application of a contact lens.

23. The contact lens device of claim 22, wherein the stem member comprises a tubular construction having a longitudinal opening aligned with an opening in the lens support.
